(19) United States
(12) Patent Application Publication
(10) Pub. No.: US 2023/0181311 A1
(43) Pub. Date: Jun. 15, 2023

(54) PLATE HAPTIC SULCUS INTRAOCULAR LENS WITH IMPROVED OPHTHALMIC VISCOSURGICAL DEVICE EVACUATION

(71) Applicant: Johnny Michel Khoury, Rancho Cucamonga, CA (US)

(72) Inventor: Johnny Michel Khoury, Rancho Cucamonga, CA (US)

(21) Appl. No.: 18/063,078

(22) Filed: Dec. 8, 2022

Related U.S. Application Data
(60) Provisional application No. 63/288,017, filed on Dec. 10, 2021.

(51) Int. Cl. A61F 2/16; A61F 9/007

(52) U.S. Cl. CPC A61F 2/16; A61F 9/00736; A61F 2002/1689; A61F 2002/0081

(57) ABSTRACT

The present disclosure describes a plate haptic sulcus intraocular lens (IOL) or implantable contact lens (ICL) comprising an optic and a haptic component and at least one open channel for improved evacuation of Ophthalmic Viscosurgical Device (OVD).

### TECHNICAL FIELD

This invention generally relates to Intraocular Lens(es) (IOL) and Implantable Contact Lens(es) (ICL), more particularly, to plate haptic sulcus IOL or ICL comprising an optic and a haptic component and at least one open channel. An exemplary lens of the invention provides for improved evacuation of Ophthalmic Viscosurgical Device (OVD).

### BACKGROUND

Plate haptic sulcus intraocular lens(es) (IOL) and Implantable Contact Lens(es) (ICL) are currently being implanted to correct refractive errors in phakic eyes.

The plate haptic sulcus IOL/ICL has a vault to minimize and/or eliminate the optic of said IOL/ICL potential contact with the eye's crystalline lens. However, such desirable vault has the potential to trap Ophthalmic Viscosurgical Device (OVD) in the space between the IOL/ICL and the crystalline lens.

The trapped OVD is non-desirable since it can potentially lead to Intraocular Pressure (TOP) elevation in the immediate post-operative period (up to approximately 48-72 hours). More importantly, it can potentially lead to a delay in the appropriate diagnosis of the rare but devastating and potentially legally blinding malignant glaucoma should it occur. (REFERENCE 1).

The current addition of an optical hole or holes is welcomed and desirable in eliminating the need of a Peripheral Iridotomy (PI) and in the prevention of pupillary block glaucoma. The description of the function of such holes or openings is limited to allowing fluid passage from the posterior to the anterior chamber of the eye.

The present invention relates to a plate haptic sulcus IOL/ICL, but not excluding other IOLs/ICLs, where there is a need to evacuate OVD trapped under the IOL/ICL and, in particular, between the IOL/ICL and the eye's crystalline lens.

A number of prior art address the subject of allowing fluid flow from the posterior to the anterior chamber of the eye by adding hole(s)/openings and hence eliminating the need for iris peripheral iridotomy(ies) and preventing pupillary block glaucoma.

US20080109078A1 describes a posterior chamber phakic intraocular lens wherein the platformed positioned arms define at least a pair of fluid pockets between the crystalline lens of the phakic lens. These fluid pockets are filled with OVD during surgical implantation. Fluid can only fill in after the OVD is cleared.

WO 2014/167425A1 describes a lens device structurally adapted to be positioned in the posterior chamber of the eye. The device comprises a generally circular optical section, two generally flat haptic structures at radially opposite sides of the optical part, and a vaulted section connecting the optical section and the haptic structures. In some embodiments, the device comprises at least one opening for allowing flow of liquid, through the device, between the posterior chamber and the anterior chamber of the eye.

WO 2016/054624A1 describes an improved posterior chamber phakic intraocular lens (PCP-IOL) having a haptic with a collar, self-adjusting struts, and lens. Additionally, a PCP-IOL as set forth herein may allow peripheral aqueous flow between anterior and posterior chambers of a patient's eye.

In the prior art, for example, US20160067035A1 describes an implantable contact lens having a central hole to provide fluid flow from the posterior to the anterior chamber of the eye.

WO 2021/152815A1 discloses a phakic intraocular lens that is implanted between the iris and the lens of an eye, wherein said phakic intraocular lens compromises a lens body disposed in the center thereof and having a hole formed therein.

In fact, no prior art clearly addresses the need or a method to evacuate OVD trapped between the plate haptic sulcus IOL/ICL and the eye's crystalline lens. The stated/described function for currently existing holes/openings is for fluid passage from posterior to anterior chamber of the eye. The purpose of adequately evacuating trapped OVD is to prevent IOP elevation related to the presence of OVD. Until the trapped OVD clears on its own which can take up to 48-72 hours, the eye is at risk of optic nerve injury, Urrets-Zavalia syndrome (REFERENCE 2), in addition to excruciating eye pain. Moreover, with OVD as a cause of immediate post-operative TOP elevation eliminated, the diagnosis of the much more serious and urgent malignant glaucoma can be entertained early on resulting in expedited appropriate treatment and a more favorable outcome with the potential to prevent legal blindness (REFERENCE 1). Moreover, the functionality of the holes/openings created by the prior art could be hindered by the presence of OVD, and only become effective when the OVD is cleared.

None of the previous art address the need or method to irrigating OVD trapped under the lens optic. Besides, the described existing holes or openings are ineffective unless the OVD is cleared from and around their passage.

### BRIEF DESCRIPTION OF THE DRAWINGS

FIG. 1 is a top view of the disclosed IOL/ICL.

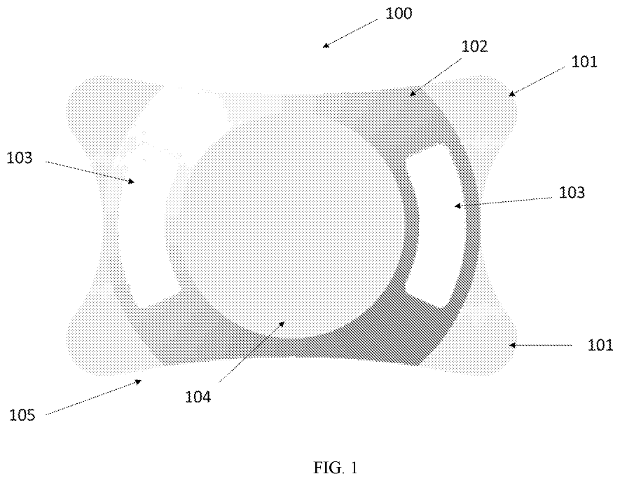

### DESCRIPTION OF THE INVENTION

The present invention discloses the addition of opening(s)/conduit(s) 103 outside the lens optic border 100, particularly in the incline/slope (vault) between the IOL's optic 104 and haptic 105. Said opening 103 should be wide enough to allow an irrigating cannula to pass through the opening freely and long enough to allow for sideways movement of said cannula to achieve effective irrigation and evacuation of OVD. The objective being for that irrigating cannula to go through the accessible opening (accessible to the entry incision) 103 and under the IOL/ICL 100 allowing the irrigating fluid to evacuate trapped OVD from all such openings 103 including the irrigating opening itself. As such one accessible opening is sufficient but not as efficient as two opposing openings 103 or more (for clarification, one such opening needs to be accessible, the others not).

In one embodiment, the openings can expand sideways and connect to become one continuous circumferential opening around the lens optic in the incline between the IOL/ICL optic 104 and haptic 105. The lumen of such arcs/conduits can be variable but at least one should be of such width as to allow the free passage of an irrigating cannula (30 gauge at least). Said lumen size should be long enough to allow sideway sweeping movement of such cannula for any sufficient irrigation and evacuation to occur. It is preferable that the irrigating cannula be marked to allow exact distance penetration under the IOL/ICL to prevent said cannula touching the crystalline lens.

The sulcus of the human eye is a relatively tight space and is a conduit for fluid to flow from the posterior to the anterior chamber of the eye. Adding an IOL/ICL 100 in the eye sulcus is sufficient to crowd the sulcus space. Moreover, Ophthalmic Viscosurgical Device (OVD) placed in the eye during surgery will hamper fluid flow until cleared.

OVD trapped in the eye will hamper fluid flow until cleared and may result in any or many several unwanted events including: 1) eye pain/can be severe and excruciating. 2) the need for topical, and/or oral, and/or intravenous medications administered frequently initially and urgently. 3) close monitoring by a qualified health care provider. 4) Urrets-Zavalia syndrome (fixed dilated pupil) resulting in permanent glare and halos. 5) pupillary block glaucoma in the immediate post-operative period; the current holes are only effective if and when clear of OVD. 6) potential delay in the appropriate diagnosis of malignant glaucoma, a potentially legally blinding disease. (REFERENCE 1).

As such, OVD used during this particular intraocular procedure is best cleared at the end of surgery to prevent any potential Intraocular Pressure (TOP) elevation caused by the retained OVD.

OVD can potentially and easily be trapped in the space between the IOL/ICL ant the crystalline lens given the anatomy and the design of the IOL/ICL, especially with the vault required to help prevent/eliminate the IOL/ICL touching the crystalline lens. Irrigating that space to clear the trapped OVD is difficult with currently existing IOL/ICL designs. There is currently no easy access to that space with the current design.

Creating/adding open channels or vents 103 to the IOL/ICL with the following criteria can provide easy access to and irrigation of the OVD trapped in the space between the IOL/ICL and the crystalline lens.

Said open channel should be wide enough to allow an irrigating cannula (at least 30-gauge cannula) to go through the opening freely allowing for irrigation under the IOL/ICL optic.

Said open channel should be long enough to allow sideway sweeping movement of such cannula for any sufficient irrigation and evacuation to occur.

The open channel should be outside the optic part of the IOL/ICL and far enough as to not interfere with the optical correction nor the optical quality of the IOL/ICL

Said channel is preferably placed in the incline/slope/vault connecting the IOL/ICL optic and haptic.

Said channel is best located in the proximal half of the IOL/ICL vault for safer irrigation (away from the crystalline lens)

One said channel (accessible to the entry incision) allowing the irrigating fluid to evacuate trapped OVD from the same channel is sufficient.

In another embodiment, at least two such channels 103 are present to allow for irrigation from one channel and evacuation from both channels. Intuitively, the more the channels or one continuous circumferential channel, the faster and more effective/complete the OVD evacuation.

Said channels can have variable width and length as dictated by the IOL/ICL design. At least one such channel should allow the irrigating cannula to freely pass through and sweep sideways to allow effective irrigation and evacuation of the OVD.

The irrigating cannula is preferable to be clearly marked to allow exact distance penetration under the IOL/ICL to prevent said cannula touching the crystalline lens.

The purpose of said built-in open channels is to facilitate the irrigation and evacuation of trapped OVD between the plate haptic sulcus IOL/ICL and the eye's crystalline lens, leading to an overall reduction in the incidence of early postoperative IOP elevation after such surgery. Hence preventing a myriad of unwanted complications and allowing for a speedy diagnosis and thus early treatment and management of other more serious causes of elevated IOP in the immediate postoperative namely malignant glaucoma.

It is also herein disclosed a preferred method for irrigating OVD from under the plate haptic sulcus IOL/ICL using the said channel/channels once the IOL/ICL is inside the eye. Said preferred method comprises the following steps:
1) Tuck the distal ends of the haptic under the iris, leaving the proximal ends of the haptic on top of the iris,
2) Enter with the irrigating cannula through the main incision and into the proximal said channel (not to exceed the marked stop sign on the cannula) and directly irrigate OVD from under the IOL/ICL. It is possible to slightly lift the IOL/ICL with the irrigating cannula while irrigating,
3) Tuck the proximal ends of the haptic under the iris,
4) Use the current method of indirect irrigation using a rightward or leftward vortex of irrigating fluid to remove OVD under the IOL/ICL through the Bernoulli Effect. Said open channels renders this method more effective in OVD removal.

Hence, such open channels allow for 1) direct irrigation under the IOL/ICL and 2) enhances the currently used indirect irrigation method (vortex of fluid and Bernoulli's Law). Combining both direct and enhanced indirect removal of OVD ensures the best outcome.

Even if direct irrigation is not applied as per surgeon's discretion, said open channels enhances the currently used indirect irrigation method (vortex of fluid and Bernoulli's Law) allowing for better and more complete OVD evacuation from under and around the ICL/IOL.

The disclosure and embodiments disclosed herein are merely for the purpose of describing the invention. No unnecessary limitations are to be implied therefrom beyond the requirement of the prior art because such terms are used for descriptive purposes and are intended to be broadly construed. Moreover, the description and illustration of the invention is an example and the invention is not limited to the exact details shown or described.

REFERENCE 1: Chanbour W A, Cherfan C G, Jarade E F, Khoury J M. Unilateral malignant glaucoma postbilateral implantable collamer lens: Effect of miotics. Middle East Aft J Ophthalmol 2019; 26(3):181-3.

REFERENCE 2. Al Habash A, Al Arfaj K, Al Abdulsalam O. Urrets-Zavalia syndrome after implantable collamer lens placement. Digit J Ophthalmol 2015; 21(3):1-11.

What is claimed is:

1. A plate haptic sulcus intraocular lens (IOL) or implantable contact lens (ICL) comprising an optic and a haptic component and at least one open channel wherein,
said open channel is wide enough to allow an irrigating cannula of at least 30-gauge to go through said channel freely allowing for irrigation under the IOL/ICL optic;
said open channel is long enough to allow sideways sweeping movement of said cannula for any sufficient irrigation and evacuation to occur;
said open channel is positioned outside the optic part of the IOL or ICL and far enough so as to not interfere with the optical correction nor the optical quality of the IOL or ICL;
said channel is preferably placed in the incline, slope and/or vault connecting the IOL or ICL optic and haptic; and
said open channel, which is accessible to the eye entry incision, allows the irrigating fluid to evacuate trapped Ophthalmic Viscosurgical Device (OVD) from the same said open channel.

2. The plate haptic sulcus IOL or ICL according to claim 1, wherein at least two channels are present to allow for irrigation from one or more channel and evacuation from two or more channels.

3. The plate haptic sulcus IOL or ICL according to claim 1, wherein said open channel is a continuous circumferential channel allowing for a faster and more effective OVD evacuation.

4. The plate haptic sulcus IOL or ICL according to claim 1, wherein said channels comprise variable width and length as dictated by the IOL or ICL design, and wherein at least one said channel allows an irrigating cannula to freely pass through and sweep sideways to allow effective irrigation and evacuation.

5. The plate haptic sulcus IOL or ICL according to claim 4, wherein said irrigating cannula is marked to allow exact safe distance penetration under the IOL/ICL to prevent said cannula touching the crystalline lens.

6. The plate haptic sulcus IOL or ICL according to claim 4, wherein said irrigating cannula is covered with a protective slippery silicone sleeve to protect with the remote but possible inadvertent touching of the crystalline lens, In a preferred embodiment, the protective slippery silicone sleeve is marked to allow exact safe distance penetration Under the IOL/ICL.

7. The plate haptic sulcus IOL or ICL according to claim 1, wherein said irrigating cannula is angled as to allow the irrigating fluid to flow parallel to the crystalline lens, which will facilitate irrigation and/or evacuation.

8. The plate haptic sulcus IOL or ICL according to claim 1, wherein said irrigating cannula comprises side port irrigation.

9. A method for irrigating OVD from under the plate haptic sulcus IOL/ICL using the at least one channel according to claim 1 once the IOL/ICL is inside the eye comprising the following steps:
tuck the distal ends of the haptic under the iris, leaving the proximal ends of the haptic on top of the iris,
enter with the irrigating cannula through the main incision and into the proximal said channel (not to exceed the marked stop sign on the cannula) and directly irrigate OVD from under the IOL/ICL, It is possible to slightly lift the IOL/ICL with the irrigating cannula while irrigating,
tuck the proximal ends of the haptic under the iris,
use the current method of indirect irrigation using a rightward or leftward vortex of irrigating fluid to remove OVD under the IOL/ICL through the Bernoulli Effect, Said open channels renders this method more effective in OVD removal.
